(19) United States
(12) Patent Application Publication
(10) Pub. No.: US 2025/0245835 A1
(43) Pub. Date: Jul. 31, 2025

(54) SYSTEMS, DEVICES, AND METHODS FOR MOTILITY-BASED DETECTION OF BACTERIA AND EVALUATION OF SENSITIVITY TO ANTIBACTERIAL AGENT

(71) Applicant: THE TEXAS A&M UNIVERSITY SYSTEM, College Station, TX (US)

(72) Inventors: Pushkar Lele, College Station, TX (US); Tanmay Lele, College Station, TX (US)

(21) Appl. No.: 19/041,936

(22) Filed: Jan. 30, 2025

Related U.S. Application Data
(60) Provisional application No. 63/626,990, filed on Jan. 30, 2024.

(51) Int. Cl. G06T 7/00; G01N 15/1433; G06T 7/20; G06V 10/778; G06V 20/69; G16H 70/20

(52) U.S. Cl. CPC G06T 7/0016; G01N 15/1433; G06T 7/20; G06V 10/7788; G06V 20/698; G16H 70/20; G06T 2207/10016; G06T 2207/10056; G06T 2207/20081; G06T 2207/30024

(57) ABSTRACT

A motility analysis system is disclosed for identifying the presence and type of bacteria via motility-based diagnostic testing. In an example, the motility analysis system receives video data of a fluid sample containing bacteria captured through a microscope, generates a two-dimensional (2D) time-projection of motile tracks of the bacteria based on a pixel-by-pixel analysis of the video data, processes the 2D time-projection of the motile tracks with a quantitative analysis model to identify a match between the 2D time-projection of the motile tracks and a plurality of predetermined 2D time-projections, and outputs data indicative of an identity of the bacteria in response to identifying the match. Certain examples can also perform such testing before and after application of antibiotics to evaluate resistance based on motility. Attachment devices for adapting computing devices into portable microscopes are also disclosed, thus providing the ability to identify bacteria on demand via motility analysis systems.

### CROSS-REFERENCE TO RELATED APPLICATIONS

This application claims the benefit of and priority to U.S. Provisional Application No. 63/626,990, filed on Jan. 30, 2024, which is incorporated by reference herein in its entirety.

### TECHNICAL FIELD

The present disclosure generally relates to systems, devices, and methods for motility-based detection of bacteria. More specifically, the present disclosure relates to systems, devices, and methods for fabricating and utilizing a motility analysis system for identifying bacteria, and/or evaluating sensitivity or resistance to antibacterial agents based on bacterial motility.

### BACKGROUND

Bacteria are abundant, wide-spread microorganisms found in every known natural environment. Certain animals and plants may be undesirably infected by invading bacteria, and efficient treatment of these bacterial infections is hindered by the present difficulties in diagnosing the types of involved bacteria. For example, a common analysis involves staining a biological sample with a stain to broadly categorize the bacteria in the sample as either Gram-positive or Gram-negative. However, this analysis is low-resolution and does not provide significant details about the infectious agent. Additional tests may include motility tests, which are often conducted in semi-solid plates. However, traditional motility tests are very slow, requiring incubation of the plate over several hours or even days in a microbiological incubator. Certain direct motility tests can be performed faster, but only provide a single datum indicating whether the bacteria are motile or non-motile. Indeed, most tests are not specific and do not help identify the particular genus or species causing a bacterial infection. A more accurate approach to identifying bacteria involves a clinical real-time polymerase chain reaction (PCR) method or a next-generation sequencing (NGS) method, which can help identify the specific infectious bacterial species. However, these protocols are time-consuming, expensive, and often only possible to execute in a dedicated facility.

Personnel in food and other industries may simply choose to skip attempts to detect or identify the bacteria. For these reasons, these personnel may choose to directly assess bacterial sensitivity to antibiotics rather than identify the particular pathogen. However, antibiotic testing also requires a long incubation period, such as more than 24 hours, and results may be unfortunately delayed by a number of days. Based on a lack of identification, some clinicians may administer broad-spectrum antibiotics to a patient when symptoms suggest a bacterial infection. Correspondingly, certain farmers may use copper compounds to broadly target bacteria, although this treatment is often impractical and may increase plant product toxicity. Additionally, these non-specific treatment approaches can result in the growth of antibiotic-resistant pathogens, which are extremely hazardous and can lead to bacteremia and sepsis.

### SUMMARY

Disclosed herein are motility analysis systems, devices, and methods for identifying the presence, type of bacteria, or both via motility-based diagnostic testing. The majority of known bacterial species are motile, with over three million species of motile bacteria identified thus far. The present disclosure provides a motility analysis system that leverages the motility of such bacterial species via unique diagnostic testing with imaging technology. The imaging-based methods are capable of identifying every type of motile bacteria species, from disease-causing to harmless species, unlike other, antigen-based tests. Additionally, the motility analysis system can evaluate any suitable fluid sample, whether from a living host (e.g., saliva, bodily fluid) or from the external environment (e.g., water tanks, aquifers, soil). Some samples can be made into fluid samples via addition of any suitable buffer solutions. To use the motility analysis system, a collected fluid sample is placed on a slide that is retained in a slide holder within view of a microscope. Movement of motile bacteria in the sample is recorded through the microscope via a camera of a computing device to acquire visual data. In some embodiments, the visual data is a motility video. The visual data is thus evaluated by an analysis software of a controller, which reports the identity of the bacteria present in the sample based on its characteristic movements. In some examples, the computing device is a hand-held device, such as a smartphone, which is transformed via a customized microscope attachment having a lens and an integrated illumination system. Such examples extend the usability of the present disclosure to a wider set of users and equip physicians, patients, and/or the general population with the ability to identify bacteria on demand. Certain examples of the motility analysis system also include an antibiotic kit, which would enable determination of whether the bacteria in the sample are antibiotic-resistant, such as by evaluating whether decreased motility is caused by one or more tested antibiotics.

The motility analysis system to detect bacterial species is uniquely designed to address the disadvantages of previous bacterial tests for multiple reasons. The motility analysis system does not require the pathogen to be cultured for long durations, unlike other motility tests, and it does not require the expense and long wait times associated with NGS or PCR diagnostics. The motility analysis system is readily available to users, such as physicians, patients, and farmers, who can perform diagnostic testing on themselves and/or their subjects/samples. For example, the motility analysis system can be provided as a kit including a copy of the analysis software and a microscope attachment, which are each utilized with a computing device to provide in-situ bacterial analysis. The motility analysis system can provide these and additional benefits to users, such as physicians, farmers, food processors, and workers in other industries. Indeed, the motility analysis system can be used to rapidly determine if samples from environments, crops, or patients are contaminated or infected by certain pathogens. As an example, physicians can use the present techniques to monitor the pathogen load in a patient's bodily fluids during a medication regime. It is also recognized herein that additional benefits are provided by the rapid evaluation of antibiotic-resistant bacteria, which can be immediately addressed as needed by escalated responses. The analysis software is trained based on a wide variety of species to be able to distinguish between different motile, pathogenic species, such as Helicobacter pylori, Escherichia coli, and Neisseria meningitidis. The training can be achieved via any suitable pattern-recognition, machine-learning, and/or artificial intelligence engines. In some examples, the analysis software can also be continuously updated or trained based on the motility data to remain fine-tuned and up to date on existing and emergent motile species.

Embodiments include systems, devices, and methods of motility analysis for bacteria identification and evaluation of sensitivity or resistance to an antibacterial agent. One such system is a motility analysis system having a memory storing processor-executable instructions and one or more processors communicatively coupled to the memory and configured to execute the processor-executable instructions from the memory. The one or more processors are configured, when executing the instructions, to receive video data of a fluid sample containing bacteria captured through a microscope, generate a two-dimensional (2D) time-projection of motile tracks of the bacteria based on a pixel-by-pixel analysis of the video data, process the 2D time-projection of the motile tracks with a quantitative analysis model to identify a match between the 2D time-projection of the motile tracks and a plurality of predetermined 2D time-projections, and output data indicative of an identity of the bacteria in response to identifying the match.

In some examples, the one or more processors are configured, when executing the instructions, to generate the 2D time-projection by determining a pixel characteristic based on a time-varying intensity of each pixel in the video data over a duration of the video data, and reshaping the pixel characteristic of each pixel to an original image size of the video data to generate the 2D time-projection. In some examples, the pixel characteristic is a pixel variance, rate of change, standard deviation, or Fourier analysis. In some examples, the pixel characteristic is determined by performing one or more mathematical or statistical operations on raw or digitally filtered intensities in each pixel, and the 2D time-projection is generated by reshaping the corresponding calculated value to the original image size. In some examples, one or more calculated values are multiplexed with additional morphological indicators of motile bacteria, including but not limited to membrane potential, cell length, cell width, cell shape, movement patterns in various viscous fluids and chemical fields, and/or immunofluorescence labels.

In some examples, the plurality of predetermined 2D time-projections are labeled with a corresponding plurality of bacteria samples, and the quantitative analysis model is trained on the plurality of predetermined 2D time-projections to distinguish and identify motile bacteria via machine learning. In some examples, the one or more processors are further configured, when executing the instructions, to receive additional video data of the fluid sample captured after application of an antibacterial compound to the fluid sample, generate an additional 2D time-projection of motile tracks of the bacteria, and output data indicative of a resistance of the bacteria to the antibacterial compound based on a comparison between the 2D time-projection and the additional 2D time-projection. In some examples, the one or more processors are further configured, when executing the instructions, to receive additional video data of the fluid sample captured after application of serum from a subject (e.g., a human, an infected human, an animal) to the fluid sample, generate an additional 2D time-projection of motile tracks of the bacteria, and output data indicative of an immune response (e.g., inflammation) based on a comparison between the 2D time-projection and the additional 2D time-projection.

In some examples, the one or more processors are further configured, when executing the instructions, to query a database of a plurality of bacterial treatments based on the identity of the bacteria, and output data indicative of a selected bacterial treatment based on the query. In some examples, the one or more processors are further configured, when executing the instructions, to receive user input indicative of an accuracy of the identity of the bacteria, and continuously train the quantitative analysis model based on the 2D time-projection and the accuracy of the identity of the bacteria.

In some examples, the motility analysis system includes a microscope attachment configured to couple to a computing device having a camera to capture the video data. The microscope attachment includes one or more of a lens to magnify images captured through the camera, a light source to illuminate the fluid sample, and a sample retaining device configured to receive an observation retainer in view of the camera and the lens. In some examples, the observation retainer includes a microfluidic device having a first trench, a second trench, and a plurality of orthogonal channels fluidly coupling the first trench and the second trench, and the plurality of orthogonal channels is sized to isolate a motile fraction of the bacteria in the first trench or the second trench for generation of the 2D time-projection. In some examples, the observation retainer includes a microscope glass slide. In some examples, the memory, the one or more processors, and a camera to capture the video are operatively coupled together within a smartphone or a tablet.

Embodiments include methods of identifying a bacteria in a sample using one of the motility analysis systems disclosed herein. One such embodiment of a method to identify a bacteria based on motility analysis includes the steps of receiving video data of a fluid sample containing bacteria captured through a microscope; generating a two-dimensional (2D) time-projection of motile tracks of the bacteria based on a pixel-by-pixel analysis of the video data; processing the 2D time-projection of the motile tracks with a quantitative analysis model to identify a match between the 2D time-projection of the motile tracks and a plurality of predetermined 2D time-projections; and transmitting data indicative of an identity of the bacteria in response to identifying the match. In some embodiments, the step of generating the 2D time-projection may further include determining a pixel characteristic based on a time-varying intensity of each pixel in the video data over a duration of the video data; and reshaping the pixel characteristic of each pixel to an original image size of the video data to generate the 2D time-projection. The plurality of predetermined 2D time-projections can be labeled with a corresponding plurality of bacteria samples. In some embodiments, the quantitative analysis model is trained on the plurality of predetermined 2D time-projections to distinguish and identify motile bacteria via machine learning. In some embodiments, the method further includes the steps of receiving additional video data of the fluid sample captured after application of an antibacterial compound to the fluid sample; generating an additional 2D time-projection of motile tracks of the bacteria; and transmitting data indicative of a resistance of the bacteria to the antibacterial compound based on a comparison between the 2D time-projection and the additional 2D time-projection. In some embodiments, the method further includes the steps of querying a database of a plurality of bacterial treatments based on the identity of the bacteria; and transmitting data indicative of a selected bacterial treatment based on the query. In some embodiments, the method further includes the steps of receiving a user input indicative of an accuracy of the identity of the bacteria; and training the quantitative analysis model based on the 2D time-projection and the accuracy of the identity of the bacteria. In some embodiments, the method further includes the steps of coupling a microscope attachment to a computing device having a camera to capture the video data.

Aspects and advantages of these exemplary embodiments and other embodiments, are discussed in detail herein. Moreover, it is to be understood that both the foregoing information and the following detailed description provide merely illustrative examples of various aspects and embodiments, and are intended to provide an overview or framework for understanding the nature and character of the claimed aspects and embodiments. Accordingly, these and other objects, along with advantages and features of the present disclosure, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. The various features of the drawings discussed below are not necessarily drawn to scale. Dimensions of various features and elements in the drawings may be expanded or reduced to more clearly illustrate embodiments of the disclosure.

FIG. 1 illustrates an example of a motility analysis system for detecting motile bacteria, according to an embodiment.

FIG. 2 illustrates an example of a motility analysis system for detecting motile bacteria, according to an embodiment.

FIG. 3 illustrates an example of a microfluidic device having a motile bacterial separator, according to an embodiment.

FIG. 4 illustrates an example of training a motility analysis system for subsequent sample identification, according to an embodiment.

FIG. 5 illustrates an example of employing a trained motility analysis system for sample identification, according to an embodiment.

FIGS. 6A-6E illustrate examples of orthogonal channels of a microfluidic device being selectively occupied by motile cells, according to various embodiments. FIG. 6A illustrates an example of orthogonal channels of a microfluidic device being selectively occupied by motile cells, including a phase contrast image shows motile cells entering the orthogonal channels, an occupancy plot indicating that 68.04±5.513% of the channels were occupied by one or more motile cells, and an illustration showing how the force developed by its flagellar bundle helps the motile cell, shown in yellow between channel walls, easily penetrate the orthogonal channel.

FIG. 6B shows images of bacterial cells in a microfluidic device, including in image of motile cells expressing eYFP that have penetrated the orthogonal channels of the microfluidic device and an image of non-motile cells expressing eCFP that are observed exclusively in the main trench.

FIG. 6C is an occupancy plot indicating that cells of the smooth-swimming mutant occupied 76.5±13.9% of the channels visualized in the yellow fluorescence channel. FIG. 6D is a cell density plot illustrating changes in the cell density per channel over one hour when the microfluidic device contained only motile cells. FIG. 6E shows a composite image that displays an overlay of the phase and fluorescent (yellow) channels for a 1:1 mixture of non-motile (cyan) and motile (yellow) cells and its corresponding occupancy plot.

FIGS. 7A-7C show optical images obtained via confocal microscopy of a microfluidic device usable in the motility analysis system, according to various embodiments. FIG. 7A is an x-y cross-section of the trench and the growth channels at the interface of PDMS and coverslip. FIG. 7B is a x-z reconstruction of 269 z-planes acquired along the height of the trench, with the surface of the trench, growth channel, and coverslip stained using DiI, a lipophilic carbocyanine dye, and the provided scale is 10 μm. FIG. 7C is an isometric three-dimensional (3D) rendition reconstructed from z-stacks and shows a section of the DiI-stained internal surfaces of the microfluidic device.

### DETAILED DESCRIPTION

The present disclosure describes various embodiments related to motility analysis systems, devices, and methods for bacteria identification and evaluation of sensitivity or resistance to antibacterial agents. For example, a motility analysis system disclosed herein leverages imaging-based processing to isolate motile bacteria in a fluid sample and identify a type or species of the bacteria based on its movement. In some examples, movement can also be analyzed before and after application of tested antibiotics to evaluate an antibiotic resistance of any surviving bacteria. The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous. The term “plurality” as used herein refers to two or more items or components. The terms “about” or “approximately” are defined as being close to as understood by one of ordinary skill in the art. In one non-limiting embodiment, these terms are defined to be within 10 percent (%), within 5%, within 1%, or within 0.5%.

The terms “removing,” “removed,” “reducing,” “reduced,” or any variation thereof, when used in the claims and/or the specification includes any measurable decrease of one or more components in a mixture to achieve a desired result. The use of the words “a” or “an” when used in conjunction with any of the terms “comprising,” “including,” “containing,” or “having,” in the claims or the specification may mean “one,” but it is also consistent with the meaning of “one or more,” “at least one,” and “one or more than one.” The terms “wt. %”, “vol. %”, or “mol. %” refers to a weight, volume, or molar percentage of a component, respectively, based on the total weight, the total volume of material, or total moles, which includes the component. In a non-limiting example, 10 grams of component in 100 grams of the material is 10 wt. % of component.

As used herein, a “motile” species is defined as any bacterial species that uses molecular motors that are powered by an endogenous or exogenous energy source to move in its environment. Such movements may include gliding, sliding, swimming, swarming, twitching, and so forth. Motile species include bacteria that swim with the aid of their flagella, bacteria that use pili-mediated twitching motility via type IV pili to move on surfaces, and bacteria that glide on surfaces using rotary molecular motors. Motile species also include bacteria that secrete polysaccharides and use their growth to cause large-scale movement of the colony, such as Bacillus subtilis. Motile bacteria detected herein may include species responsible for any number of conditions in animals or plants, including stomach cancer, meningitis, sexually transmitted diseases, gastrointestinal disease, lung infection, eye infection, Fire blight, citrus infections, flesh-eating bacteria, and so forth.

There is an unmet demand for rapid, accessible, and efficient detection and analysis of bacterial pathogens. Certain first-line characterizations utilize serotyping or antibody-based detection, which involves a long turnaround time and expensive antisera, reagents, and storage methods. Other phenotyping techniques, such as those involving procalcitonin or C-reactive protein, cannot identify the particular pathogen involved. Although genotyping provides a high accuracy, the testing requires several days, involves expensive laboratory equipment and dedicated personnel. Indeed, each of the above-described techniques are slow, laborious, expensive, and thus impractical for use in continually monitor pathogens or repeatedly analyzing different samples, such as during routine treatment or epidemics. These techniques are also insufficient for detecting antibiotic resistance in the sampled bacteria. As such, further developments for diagnostic testing to provide detailed and rapid identification of pathogens, such as bacterial species, is desired.

Disclosed herein are motility analysis systems, devices, and methods for identifying the presence, type of bacteria, or both via motility-based diagnostic testing. The majority of known bacterial species are motile, with over three million species of motile bacteria identified thus far. The imaging-based methods are capable of identifying every type of motile bacteria species, from disease-causing to harmless species, unlike other, antigen-based tests. Additionally, the motility analysis system can evaluate the presence or absence of bacteria in any suitable fluid sample. For example, the sample can be from a living host (e.g., saliva or other bodily fluid) or an agricultural product (such as a vegetable, a leaf, or a branch) or a food product (such as meat or salad), or from the external environment (e.g., water tanks, aquifers, or soil). A non-fluid sample can be processed into a fluid sample by addition of any suitable solution, such as a buffer. To use the motility analysis system, a collected fluid sample is placed on a slide that is retained in a slide holder within view of a microscope. Movement of motile bacteria in the sample is recorded through the microscope via a camera of a computing device to acquire visual data. In some embodiments, the visual data is a motility video. The visual data is thus evaluated by an analysis software of a controller, which reports the identity of the bacteria present in the sample based on its characteristic movements. In some examples, the computing device is a hand-held device, which is transformed via a customized microscope attachment having a microscope lens and an integrated illumination system. Examples of the computing devices include, without limitation, a smartphone, a personal digital assistant device, a tablet, a mobile phone, or such portable communication devices. These devices extend the usability of the present disclosure to a wider set of users and equip physicians, patients, and/or the general population with the ability to identify bacteria on demand.

The terms “subject” and “patient” are used interchangeably herein. None of the terms are to be interpreted as requiring the supervision of a medical professional (e.g., a doctor, nurse, physician's assistant, orderly, hospice worker). As used herein, a subject may be any animal, including mammals (e.g., a human or non-human animal) and non-mammals. In one embodiment, the subject is a human.

The motility analysis system is readily available to users, such as physicians, patients, and farmers, who can perform diagnostic testing on themselves and/or their subjects/samples. For example, the motility analysis system can be provided as a kit. Such a kit would include a copy of the analysis software and a microscope attachment, which are each utilized with a computing device to provide in-situ bacterial analysis. The motility analysis system can provide these and additional benefits to users, such as physicians, farmers, food processors, and workers in other industries. Indeed, the motility analysis system can be used to rapidly determine if samples from the environments, crops, or patients are contaminated or infected by certain pathogens. As an example, physicians can use the present techniques to monitor the pathogen load in a patient's bodily fluids during a medication regime. It is also recognized herein that additional benefits are provided by the rapid evaluation of antibiotic-resistant bacteria, which can be immediately addressed as needed by escalated responses. Certain examples of the motility analysis system also include an antibiotic kit that contains one or more antibacterial agents (such as antibiotics). Such kits would enable determination of whether the bacteria in the sample are resistant to such agents by evaluating whether decreased motility is caused by one or more tested agents. The analysis software is trained based on a wide variety of species to be able to distinguish between different motile, pathogenic species, such as Helicobacter pylori, Escherichia coli, and Neisseria meningitidis. The training can be achieved via any suitable pattern-recognition, machine-learning, and/or artificial intelligence engines. In some examples, the analysis software can also be continuously updated or trained based on the motility data to remain fine-tuned and up to date on existing and emergent motile species.

Embodiments include systems, devices, and methods of motility analysis for bacteria identification and/or evaluation of sensitivity or resistance to antibacterial agents. One such system is a motility analysis system having a memory storing processor-executable instructions and one or more processors communicatively coupled to the memory and configured to execute the processor-executable instructions from the memory. The one or more processors are configured, when executing the instructions, to receive video data of a fluid sample containing bacteria captured through a microscope, generate a two-dimensional (2D) time-projection of motile tracks of the bacteria based on a pixel-by-pixel analysis of the video data, process the 2D time-projection of the motile tracks with a quantitative analysis model to identify a match between the 2D time-projection of the motile tracks and a plurality of predetermined 2D time-projections, and output data indicative of an identity of the bacteria in response to identifying the match. “Video data” refers to a collection of visual observations of the sample as received by or supplied to the computing device.

In some examples, the one or more processors are configured, when executing the instructions, to generate the 2D time-projection by determining a pixel characteristic based on a time-varying intensity of each pixel in the video data over a duration of the video data, and reshaping the pixel characteristic of each pixel to an original image size of the video data to generate the 2D time-projection. In some examples, the pixel characteristic is a pixel variance, rate of change, standard deviation, or Fourier analysis. In some examples, the pixel characteristic is determined by performing one or more mathematical or statistical operations on raw or digitally filtered intensities in each pixel, and the 2D time-projection is generated by reshaping the corresponding calculated value to the original image size. In some examples, one or more calculated values are multiplexed with additional morphological indicators of motile bacteria, including but not limited to membrane potential, cell length, cell width, cell shape, movement patterns in various viscous fluids and chemical fields, and/or immunofluorescence labels.

In some examples, the plurality of predetermined 2D time-projections are labeled with a corresponding plurality of bacteria-containing samples, and the quantitative analysis model is trained on the plurality of predetermined 2D time-projections to distinguish and identify motile bacteria via machine learning. In some examples, the one or more processors are further configured, when executing the instructions, to receive additional video data of the fluid sample captured after application of an antibacterial compound to the fluid sample, generate an additional 2D time-projection of motile tracks of the bacteria, and output data indicative of a resistance of the bacteria to the antibacterial compound based on a comparison between the 2D time-projection and the additional 2D time-projection. In some examples, the one or more processors are further configured, when executing the instructions, to receive additional video data of the fluid sample captured after application of serum from a subject (e.g., a human, an infected human, or an animal) to the fluid sample, generate an additional 2D time-projection of motile tracks of the bacteria, and output data indicative of an immune response (e.g., inflammation) based on a comparison between the 2D time-projection and the additional 2D time-projection.

In some examples, the one or more processors are further configured, when executing the instructions, to query a database of a plurality of bacterial treatments based on the identity of the bacteria, and output data indicative of a selected bacterial treatment based on the query. In some examples, the one or more processors are further configured, when executing the instructions, to receive user input indicative of an accuracy of the identity of the bacteria, and continuously train the quantitative analysis model based on the 2D time-projection and the accuracy of the identity of the bacteria and/or reactivity to the antibacterial agent.

In some examples, the motility analysis system includes a microscope attachment configured to couple to a computing device having a camera to capture the video data. The microscope attachment includes one or more of a lens to magnify images captured through the camera, a light source to illuminate the fluid sample, and a sample retaining device configured to receive an observation retainer in view of the camera and the microscope lens. In some examples, the observation retainer includes a microfluidic device having a first trench, a second trench, and a plurality of orthogonal channels fluidly coupling the first trench and the second trench. The plurality of orthogonal channels is sized to isolate a motile fraction of the bacteria in the first trench or the second trench for generation of the 2D time-projection. In some examples, the observation retainer includes a microscope glass slide. In some examples, the memory, the one or more processors, and a camera to capture the video are operatively coupled together within a smartphone or a tablet.

The presently disclosed motility analysis system addresses these shortcomings via a unique integration of machine learning processes with advances in the understanding of bacterial motility. For example, the motility analysis system implements image analysis and machine learning to identify the type of motile bacteria from microscopy data of a sample fluid for rapid diagnostic and screening purposes. Examples of the system also includes a custom-made microscope attachment including a lens and a slide holder, which are coupled to a handheld device, such as a smartphone or a dedicated diagnostic computing device. The resulting device can easily and efficiently collect video data (e.g., a video, a sequence of timestamped still images or frames) of any fluids under evaluation, including saliva, genital fluids, or stool samples from animals, including humans, as well as fresh and saltwater bodies, food and vegetation, animal products, fluidized soil samples, and so forth. The motility analysis system implements a quantitative analysis model or algorithm to distinguish between different motile bacterial species. In some examples, the determination of a bacterial species can be performed on demand, solely based on microscopy data obtained over a few seconds to minutes. The motility analysis system thus enables patients or physicians to perform rapid testing on their samples within a few minutes and identify the pathogen accurately.

In more detail, the motility analysis system can utilize the quantitative analysis model to convert video data of the sample into two-dimensional (2D) projections of motile tracks (e.g., motility characteristics, motility fingerprints). In some examples, the duration of the video data is in a range between about 0.5 and 50, 0.5 and 120, 1 and 120, 1 and 60, 1 and 30 seconds, or any other suitable duration. In some examples, the frame rate at which the video data is acquired is in a range between about 10 and 500, 0.1 and 10, 0.001 and 1 hertz (Hz). In some examples, multiple independent 2D time-projections of the same sample can be obtained by performing a frequency sweep for frame rates from 0.001 to 500 Hz during acquisition, to identify different bacterial species in the same sample based on differences in their motility speeds. The motility analysis system converts the video data into 2D projections of motile tracks by calculating a pixel characteristic, such as pixel variance, rate of change, standard deviation, or Fourier analysis, obtained by performing various mathematical operations on the time-varying intensities recorded by each pixel in the image over the duration of the video data. In some examples, various digital filters or spatial band-passing are deployed to improve the quality of the incoming or processed signal. The motility analysis system then reshapes the calculated pixel characteristics to the original image size to generate the 2D time-projection of motility. This approach eliminates non-motile bacteria in the video data, leaving behind only the trajectories of the motile cells. Multiple 2D time-projection of motile cells and the corresponding frame rates are thus provided as inputs to a machine learning engine to train the machine learning engine to distinguish and identify motile bacteria based on 2D time-projections and the frame rates, thereby forming the quantitative analysis model for future use. As such, newly collected video data of a sample can be converted into 2D time-projections and provided to the machine learning engine, which identifies one or more bacterial species in the sample based on its training.

In addition to identifying the type of bacteria within a few seconds or minutes, the quantitative analysis model and the microscope attachment is combined with antibiotic test kits, in some examples. The antibiotic test kits enable rapid determination of whether there are any antibiotic-resistant bacteria present in the samples after a tested antibiotic is added. That is, the motility analysis system can screen antibiotics against the motile species to help identify antibiotic resistant bacteria in samples. The motility analysis system can also screen whether silver compounds, copper compounds, photocatalytic treatment, immune cells, and other forms of antibacterial treatments can work effectively against the sampled species. Antibiotic screening and testing are based on an evaluation of whether any cells retained motility, which indicates resistant bacteria, or whether the cells do not retain motility, which indicates non-resistant bacteria.

Antibiotic resistance in bacterial pathogens is increasingly posing considerable clinical and economic challenges, which are addressed by the presently disclosed motility analysis system. The development of resistance in susceptible bacteria can be classified into two broad categories: genotypic and phenotypic. Genotypic resistance involves the acquisition of resistance genes or mutations that protect against antibiotics and can be passed down to future generations. Phenotypic resistance encompasses cellular adaptations to antibiotic stress, without genetic changes. An example of phenotypic resistance is bacterial persistence, which can either spontaneously emerge or be induced by stress. Unlike genotypic resistance, phenotypic resistance is transient and less likely to be inherited. Adapted bacterial cells tolerate lethal antibiotics without proliferation and can resume growth once the antibiotic is removed. The presence of a tolerant pool of cells increases the likelihood of eventual emergence of genotypic resistance in the surviving bacteria. Accordingly, the motility analysis system can integrate a cell-level evaluation of whether any antibiotic resistance is displayed by sampled bacteria, thereby enabling escalated responses to address such resistance rapidly and effectively, while preventing development of genotypic modifications. The motility analysis system can analyze samples from a patient over time to evaluate whether the immune system of the patient produces an innate response capable of fighting common infections or, alternatively, is deficient in some manner.

Embodiments include methods of identifying a bacteria in a sample using one of the motility analysis systems disclosed herein. One such embodiment of a method to identify a bacteria based on motility analysis includes the steps of receiving video data of a fluid sample containing bacteria captured through a microscope; generating a two-dimensional (2D) time-projection of motile tracks of the bacteria based on a pixel-by-pixel analysis of the video data; processing the 2D time-projection of the motile tracks with a quantitative analysis model to identify a match between the 2D time-projection of the motile tracks and a plurality of predetermined 2D time-projections; and transmitting data indicative of an identity of the bacteria in response to identifying the match. In some embodiments, the step of generating the 2D time-projection may further include determining a pixel characteristic based on a time-varying intensity of each pixel in the video data over a duration of the video data; and reshaping the pixel characteristic of each pixel to an original image size of the video data to generate the 2D time-projection. The plurality of predetermined 2D time-projections can be labeled with a corresponding plurality of bacteria samples. In some embodiments, the quantitative analysis model is trained on the plurality of predetermined 2D time-projections to distinguish and identify motile bacteria via machine learning. In some embodiments, the method further includes the steps of receiving additional video data of the fluid sample captured after application of an antibacterial compound to the fluid sample; generating an additional 2D time-projection of motile tracks of the bacteria; and transmitting data indicative of a resistance of the bacteria to the antibacterial compound based on a comparison between the 2D time-projection and the additional 2D time-projection. In some embodiments, the method further includes the steps of querying a database of a plurality of bacterial treatments based on the identity of the bacteria; and transmitting data indicative of a selected bacterial treatment based on the query. In some embodiments, the method further includes the steps of receiving a user input indicative of an accuracy of the identity of the bacteria; and training the quantitative analysis model based on the 2D time-projection and the accuracy of the identity of the bacteria. In some embodiments, the method further includes the steps of coupling a microscope attachment to a computing device having a camera to capture the video data. The microscope attachment contains a sample retaining device configured to receive an observation retainer.

In some embodiments, the observation retainer contains a microfluidic device having a first trench, a second trench, and a plurality of orthogonal channels fluidly coupling the first trench and the second trench. The plurality of orthogonal channels can be sized to isolate a motile fraction of the bacteria in the first trench or the second trench for generation of the 2D time-projection.

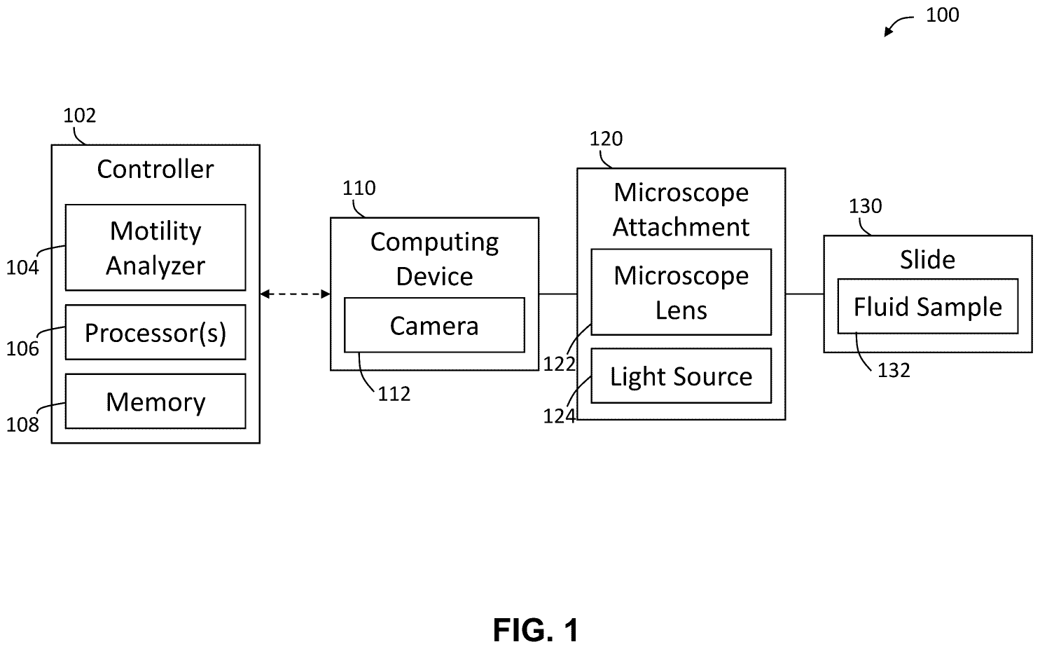

Various examples are provided herein of the motility analysis systems that facilitate convenient identification of bacteria and/or any sensitivity or resistance to any antibacterial agent. For example, FIG. 1 illustrates a motility analysis system 100 having a controller 102, a computing device 110, a microscope attachment 120, and a slide 130 (e.g., observation retainer, sample display device) that is provided with a fluid sample 132. The computing device 110 can be any suitable device having a camera 112 capable of capturing or recording video data. In some examples, the computing device 110 is a smartphone, tablet, or another handheld device. The camera 112 can collect and store video data at any suitable frame rate, such as at least 24 or 30 frames per second, in some examples. The camera 112 of certain examples includes one or more Complementary Metal-Oxide-Semiconductor (CMOS) active-pixel image sensors or charge-coupled device (CCD) type cameras.

The microscope attachment 120 includes one or more components to adapt the camera 112 of the computing device 110 for the capture of microscopic images and/or videos. In the illustrated example, the microscope attachment 120 includes a microscope lens 122 and a light source 124 (e.g., illumination system). The microscope lens 122 magnifies the images taken by the camera 112 to a degree suitable for monitoring and detecting bacteria. For example, the microscope lens 122 can magnify images through the camera 112 by a factor of about 100, 200, 300, 400, 600, 700, 800, or more. The light source 124 can include any suitable number and arrangement of light-emitting diodes (LED) or other components that emit light in a spectrum suitable for use in microscopy. The microscope attachment 120 is manufactured with any suitable frame, housing, or coupling mechanism that retains the microscope lens 122 and the light source 124 in a fixed position relative to the camera 112 of the computing device 110. In some examples, the microscope attachment 120 is 3D printed, injection molded, or otherwise readily manufacturable. The microscope attachment 120 can be designed to fit any suitable handheld or portable device with image capturing abilities. Some examples of the microscope attachment 120 are adjustable to enable a single component to couple to computing devices 110 having a variety of sizes or form factors.

Additionally, the microscope attachment 120 includes a slide holder, clip, coupling, or retainer to retain the slide 130 proximate or adjacent to the microscope lens 122. With this arrangement of components, a user of the motility analysis system 100 can collect the fluid sample 132, add the fluid sample 132 to the slide 130, and attach the slide 130 to the microscope attachment 120. The computing device 110 can therefore collect video data of the fluid sample 132, through the microscope lens 122 with the camera 112, and provide the video data to the controller 102. In some examples, the microscope attachment 120 therefore transforms the computing device 110 into a specialized microscope for inspecting and analyzing bacteria. However, in some examples, the presently disclosed methods for motility analysis can be performed with a stand-alone or desktop microscope having a camera.

The controller 102 includes specialized programming, such as a software application, which analyzes the received video data and outputs an identification of any detected bacteria in the fluid sample 132. In the illustrated example, the controller 102 includes a motility analyzer 104, which is executed or managed by one or more processors 106 and at least one memory 108. The memory 108 stores or includes instructions that are executable by the processor 106. In some examples, the controller 102 or the motility analyzer 104 thereof includes both a set of scripts in a numeric computing code as well as code for a quantitative analysis model, which is developed and trained via machine learning and/or artificial intelligence (AI). Certain examples herein use MATLAB software from Mathworks Inc., USA as the numeric computing code and use AI software from Landing AI, USA.

The motility analyzer 104 can generate 2D time-projections of motile tracks in video data to provide a representative picture of movement within the fluid sample 132 for comparison and identification. For example, the motility analyzer 104 of certain examples receives video data of the fluid sample 132 and generates a 2D time-projection of motile tracks of the bacteria based on a pixel-by-pixel analysis of the video data. Then, the 2D time-projection is processed via a quantitative analysis model of the motility analyzer 104 to identify any suitable matches between the 2D time-projection and a set of predetermined or labeled 2D time-projections associated with identified bacteria. The motility analyzer 104 thus provides the identity of one or more bacterial species in the fluid sample 132 based on any corresponding matches.

The motility analyzer 104 performs one or more mathematical operations to calculate individual pixel characteristics. For example, the pixel characteristic can be a variance, rate of change, standard deviation, and/or a Fourier analysis, with any suitable amount of contrast enhancement. As such, the motility analyzer 104 can generate and analyze 2D motility tracks for pathogen identification, without the use of any particle tracking that incurs more processing, time, and error generation. Indeed, the motility analysis system 100 provides an efficient and comprehensive diagnostic system. For example, a fluid sample is loaded into an observation retainer (e.g., glass microscope slide, microfluidic device, motile bacteria separator (MBS)) and illuminated in a darkfield or phase illumination mode, and signals are relayed to a computing device via a set of collection lenses to collect suitable image and/or video data. The motility analyzer 104 integrates artificial intelligence-based bacterial phenotyping to process images and convert spatiotemporal signals into motility fingerprints (2D data). The motility analyzer 104 compares these motility fingerprints against a database of previously identified motility fingerprints to identify the pathogen.

In some examples, the camera 112 includes certain specialized processing hardware. For example, a Prophesee camera (Paris, FR) can be used to obviate certain calculation steps, such as determining the pixel characteristic (e.g., variance, rate of change, standard deviation, Fourier analysis) per pixel. For example, the specialized camera can monitor pixels that respond independently and asynchronously to changes in the recorded photon intensities to automatically generate motility fingerprints. This recordation obviates the demand for recording videos and computing variance or other pixel characteristics altogether when generating motility fingerprints used by the motility analyzer 104.

The controller 102 leverages multiple technologies to convert imaging data of motile bacteria into input for a machine learning algorithm or quantitative analysis model and to train the algorithm to identify the type of motile bacteria. These methods utilize the identified differences (e.g., motility characteristics) in active movement of different bacterial species to detect and identify bacteria in fluid samples. The motility analysis system 100 can distinguish any motile species of human, animal, and plant pathogens. For example, experimental testing was performed to evaluate the ability of the motility analysis system 100 to distinguish between various samples, such as a first species of H. pylori and a second species of E. coli. Using the methods and techniques disclosed herein, the motility analysis system 100 performed at a 100% success rate in distinguishing between these two species based on image analysis of 2 second microscopy data.

Additionally, certain examples of the controller 102 include software programming or mobile applications that can be installed on the same device as the microscope attachment 120, thereby providing integrated motility analysis that can report on identified types of bacteria. In such embodiments, the motility analysis system 100 provides a comprehensive and portable tool for performing rapid bacterial diagnostics, based on uniquely trained machine learning and/or artificial intelligence engines. In an example, a kit can be produced or provided that includes access to or a copy of the software application for image analysis and/or machine learning application code, as performed by the controller 102. In another example, a kit includes the software application along with the microscope attachment 120, a set of slides 130 or other suitable observation retainers (e.g., microfluidic devices), buffer solutions (e.g., phosphate saline buffer solutions), and/or antibiotic kits and antibacterial compounds for antibiotic-resistance tests.

In some examples, the motility analysis system 100 also recommends a specific treatment based on the identified types of bacteria. The treatment can include any one or more antibiotics or metal-based (e.g., copper, silver) treatments specifically identified based on their specific efficacy in targeting the identified species. Examples of the motility analysis system 100 also provide testing for the presence of antibiotic resistant bacteria in any motile bacterial species. As one example, the motility analysis system 100 can analyze the motility both before and after application of a particular antibiotic to quantify the effect of the antibiotic. This small-scale testing on sample fluids provides benefits over the uninformed use of broad-spectrum antibiotics, which contribute to bacterial tolerance.

As used herein, a “processor”, processing resource, or processing circuitry may be a plurality of processors connected together in communication with an electronic communications network. In other embodiments, the processors may be a group of graphical processing units configured to work in parallel as a graphics processing unit (GPU) cluster. A processor may include a single processor device and/or a plurality of processor devices, such as distributed processors. Additionally, a processor may be any suitable processor capable of executing/performing instructions. For example, a processor may include a central processing unit (CPU), a semiconductor-based microprocessor, a GPU, a field-programmable gate array (FPGA) to retrieve and execute instructions, and/or a real-time processor (RTP) that carries out program instructions to perform the basic arithmetical, logical, and input/output operations required to support the motility analysis system 100. A processor may include code, such as processor firmware, a protocol stack, a database management system, an operating system, or a combination thereof, that creates an execution environment for program instructions. Processes and logic flows described herein may be performed by one or more programmable processors executing one or more computer programs to perform functions by operating on input data and generating corresponding output.

In an example, the memory 108 may be a non-transitory machine-readable storage medium. As used herein, a “machine-readable storage medium” may be any electronic, magnetic, optical, or other physical storage apparatus or cyber-physical separation storage to contain or store information such as executable instructions, data, and the like. For example, any machine-readable storage medium described herein may be any of random-access memory (RAM), volatile memory, non-volatile memory, flash memory, a storage drive such as hard drive, a solid-state drive, any type of storage disc, and the like, or a combination thereof. As noted, the memory 108 may store or include instructions executable by the processor 106. Accordingly, the processor 106 may execute machine-readable instructions of the memory 108 to perform the various detections and evaluations of the motility analysis system 100 disclosed herein.

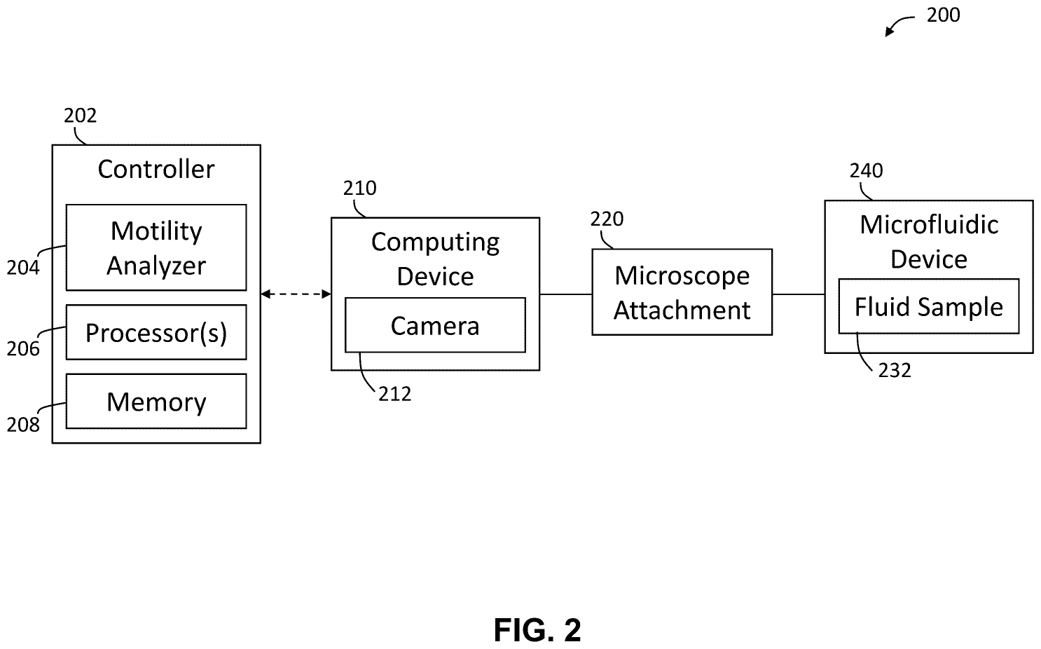

FIG. 2 illustrates a motility analysis system 200 having a controller 202, a computing device 210, a microscope attachment 220, which are similar to the corresponding components discussed above with reference to FIG. 1. However, in place of the slide, the illustrated example of the motility analysis system 200 utilizes a microfluidic device 240 (e.g., observation retainer, sample display device) to hold the fluid sample 232 under inspection. The microfluidic device 240 is a narrow-channel microfluidic device or mother machine that can achieve up to 100% separation of motile cells from a mixture of motile and non-motile bacteria, as discussed with reference to later figures. In some examples, the microfluidic device 240 can be utilized to isolate the motile cells in a sample for more efficient detection and identification by the motility analyzer 204.

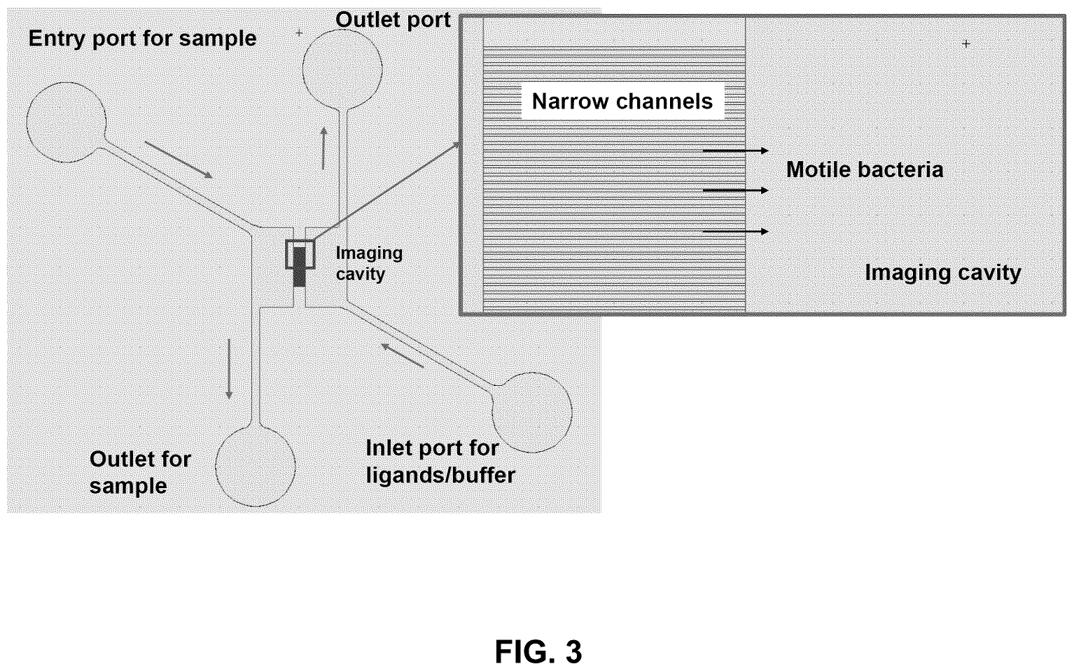

The microfluidic device 240 includes two main trenches, each with an outlet and an inlet, connected by a set of orthogonal channels that each have a width that is similar to a size of individual bacterial cells. For example, the sample is introduced in a first trench, and the motile bacteria propel themselves through the narrow channels and into the second trench. Non-motile bacteria and objects larger than the channels are unable to cross the channels into the second trench. As such, in certain examples all imaging and/or calculations are performed on only the second trench, which includes only motile bacteria to be identified. An example of this microfluidic device 240 is illustrated in FIG. 3 and further discussed below.

Single-cell assays can be performed based on their increased suitability for providing population-level data over both conventional bulk separation methods and population immobilization on glass slides for microscopy. In the present disclosure, the population is loaded into the microfluidic device 240 in the absence of external forces to cause only motile cells to traverse the orthogonal channels into an observation trench. The heightened viscous drag inside the channels prevented the entry of the immotile cells, thereby causing the orthogonal channels to be occupied and traversed exclusively by motile cells. This loading procedure is distinguished over previous uses of certain microfluidic devices, in which researchers apply centrifugal forces to load cells into the channels of the device before conducting measurements. Indeed, such centrifugal-based loading causes the motile and non-motile cells to be indistinguishable once they have entered the channels, and such devices do not provide the efficient separation of the presently closed microfluidic device 240 having two trenches fluidly coupled by a set of narrow channels.

In the illustrated example, the camera 212 of the computing device 210 can record videos or capture video data as the motile cells enter, dwell in, and/or exit the channels of the microfluidic device 240. In some examples, a user can introduce a sample into the microfluidic device 240, wait a time period for at least a portion of the motile cells to enter the channels, and then introduce a buffer solution into the main trench of the microfluidic device 240 to remove all immotile cells. After the immotile cells are removed, video data can thus be captured of only the motile cells, which may remain in the channels and/or reenter the main trench. The motile cells will generally move in characteristic patterns having particular motility characteristics, such as motile tracks, directions, speeds, and so forth, which are identified by the motility analyzer 204. As noted above, the motility analyzer 204 can generate 2D time-projections of motile tracks in video data to provide a representative picture of movement for comparison and identification.

FIG. 3 illustrates an example of a microfluidic device that can be integrated into the motility analysis system discussed herein. In some examples, the microfluidic device is a bioseparator microfluidic device and/or an integrated microscopy device. In some examples, the microfluidic device is or includes a motile bacterial separator. For example, the motile bacteria separator includes two main trenches that are fluidly connected by orthogonal channels. A first main trench (e.g., entry cavity) is used to receive a fluid sample, from which motile bacteria travel through the thin or narrow channels to enter a second main trench (e.g., imaging cavity). The narrow channels prevent immotile bacteria as well as other objects larger than the channel openings (e.g., human cells, immune cells, fungi) from entering the imaging cavity. Motility is thus recorded in the imaging cavity that specifically and exclusively extracts motile bacteria from a sample.

In some examples, the motile bacteria separator is incorporated in an integrated microscopy device. The motile bacteria separator relays only motile bacteria to the imaging cavity by physically excluding the non-bacterial objects. This significantly improves the accuracy of the machine learning-based identification tool as the imaging does not record cells other than motile bacteria. Such operations also obviate the demand for dedicated or trained personnel to perform microscopy-related experiments with multiple sample loading steps. In the illustrated example, the integrated microscopy device also includes additional ports to increase the convenience of its use. For example, an entry port is fluidly coupled to the first main trench to supply a fluid sample thereto. Motile bacteria in the sample can traverse the channels into the second main trench for imaging therein. The integrated microscopy device includes a first outlet port fluidly coupled to the first main trench and includes a second outlet port fluidly coupled to the second main trench. Through these outlet ports, the respective sample portions of the main trenches can be removed to prepare the integrated microscopy device for subsequent use. The integrated microscopy device also includes an inlet port to receive any ligands and/or buffers and direct the additives to the motile bacteria separator or its second main trench.

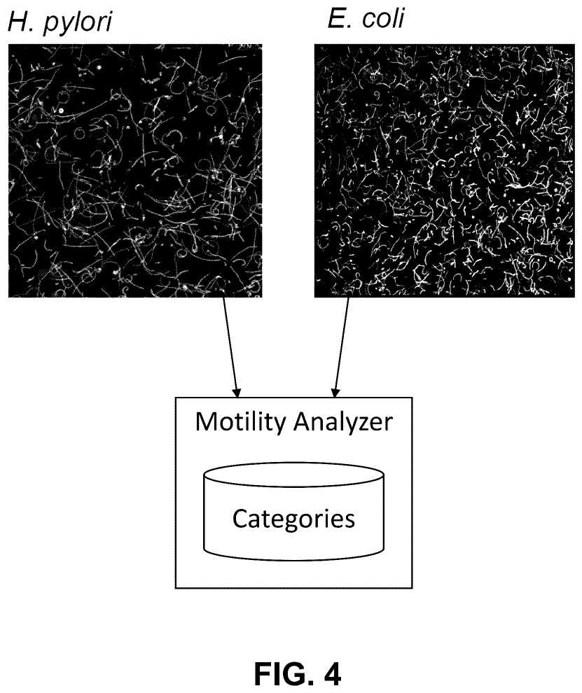

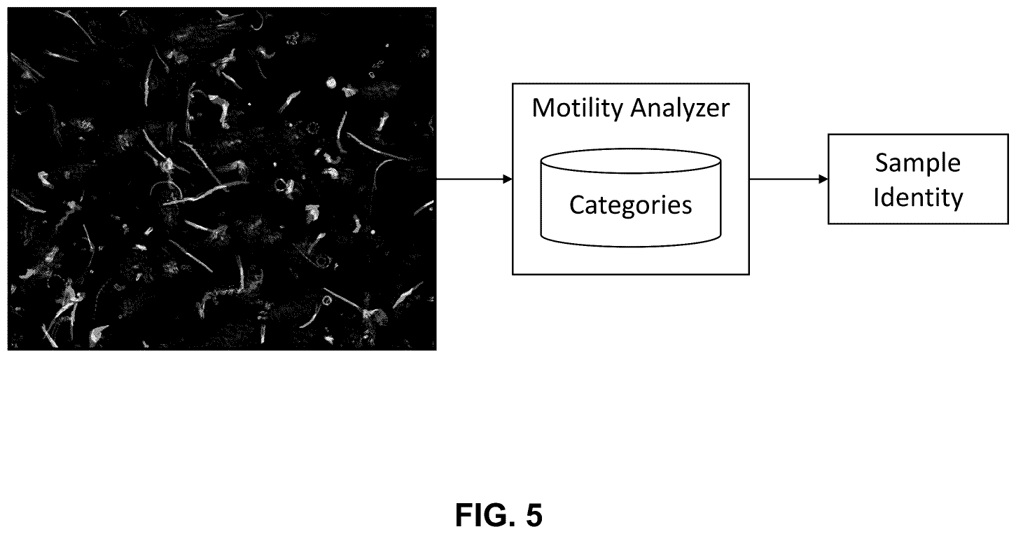

As described herein, the motility analysis system automatically generates motility fingerprints or 2D time-projections from microscopy signals, illustrated with respect to FIGS. 4 and 5. For example, FIG. 4 illustrates an example of training the motility analyzer of a motility analysis system. The motility analysis system is trained to generate and expand categories based on training performed with labeled or identified motility fingerprints. Inputs are provided to the motility analyzer, such as samples that are labeled or tagged with the bacterial species therein. For example, motility fingerprints, motility tracks, and/or 2D time-projections of movement for both H. pylori and E. coli are illustrated, as generated by the present system and methods. Each species or subset of motile bacteria perform representative motions or actions that are representative of their distinct identity and captured in the motility fingerprints. The motility analyzer can then generate categories for each of the labeled species and their motility fingerprints to enable their identification in subsequently processed samples. The motility analyzer can be trained to detect the majority of known bacteria, including common species such as H. pylori, E. coli, P. aeruginosa, S. enterica, V. cholerae, C. jejuni, C. difficile, N. gonorrhea, V. parahaemolyticus, and P. mirabilis,

FIG. 5 illustrates an example of employing the motility analyzer of the motility analysis system for sample identification. The motility analyzer receives video data of an unknown sample, translates video data into a standardized format or motility fingerprint, and processes the motility fingerprint to identify any matches to the categories generated based on previous training. In some examples, matches are determined based on a threshold probability, such as at least a 70, 80, 90, 95 percent likelihood that the sample matches a category. In illustrated example, the motility tracks are a match with the motility tracks of H. pylori, and the motility analyzer outputs data indicative of the identity of the species based on the match. As such, a motility fingerprint of an unknown sample can be benchmarked via machine learning with the trained model, and the motility analysis system provides a decision indicative of the identity of the bacteria in the unknown sample.

In another example, the detection software utilized herein can distinguish various fluid samples to identify any content therein of stomach-cancer causing bacteria (H. pylori), and pathogens that cause urinary tract infections (E. coli) and lung infections (P. aeruginosa). In some examples, the identity is also determined based on the combination of multiple morphological markers. For example, the motility fingerprint analysis can be analyzed with one or more morphological markers determined from the video data in a multiplexed approach.

Accordingly, the motility analyzer digests input motility fingerprints with machine learning/artificial intelligence algorithms that compare the inputs against training datasets of motility fingerprints for different bacterial species. The models automatically match and categorize the fingerprints to identify the bacterial species via high throughput determinations that evaluate large populations rather than individual bacterial cells. Such benchmarking considers the relative motion of bacterial cells because the fingerprints contain differential bacterial movement in a population. The motility analyzer can be used in portable imaging tools because minimal human intervention is used to obtain or benchmark the fingerprints. These tools can, therefore, be deployed by any non-expert anywhere to identify the bacteria in any fluid sample.

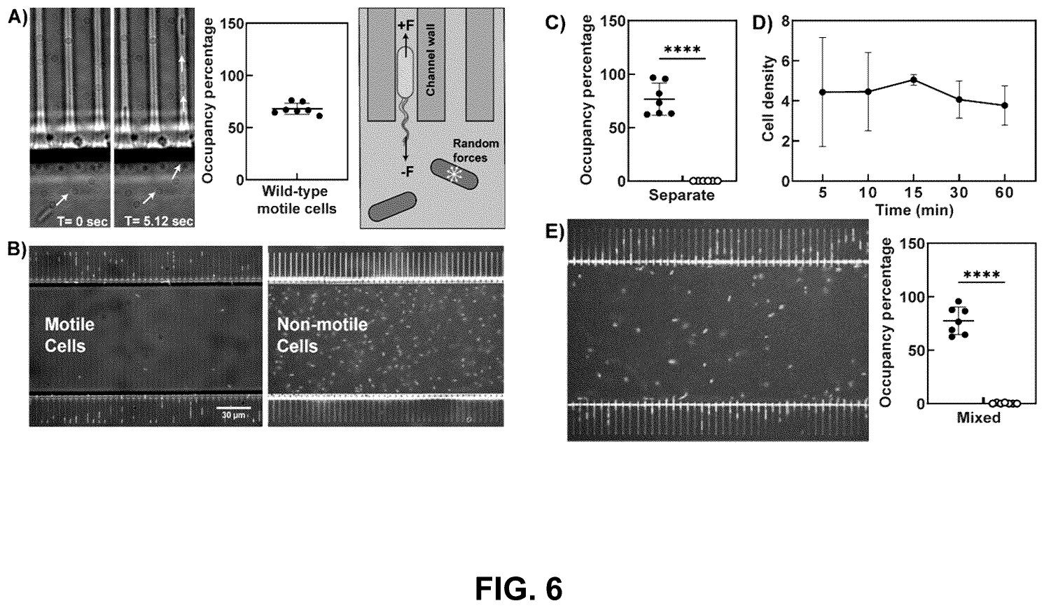

As discussed herein, various experimental testing was performed to provide the evidence, techniques, quantitative image processing, and algorithm training utilized by the motility analysis system. In more detail, FIG. 6A illustrates an example of orthogonal channels of a microfluidic device being selectively occupied by motile cells. In the left panel, a phase contrast image shows motile cells entering the orthogonal channels of the microfluidic device. The white arrows indicate cell trajectories or motile tracks over a monitored time period of 5.12 seconds. The middle panel provides an occupancy plot indicating that, of the total channels inspected (n=622 channels), 68.04±5.513% of the channels were occupied by one or more motile cells of the wild-type strain. As illustration, the right panel shows how the force developed by its flagellar bundle helps the motile cell, shown in yellow between channel walls, easily penetrate the orthogonal channel. The non-motile cells shown in blue are acted upon by smaller magnitude of Brownian forces in random directions, preventing their entry into the channels. As another example, FIG. 6B shows images of bacterial cells in a microfluidic device. In the left panel, motile cells expressing eYFP have penetrated the orthogonal channels of the microfluidic device. Additionally, very few cells continued swimming in the main trench. In the right panel, non-motile cells expressing eCFP can be observed exclusively in the main trench.

FIG. 6C is an occupancy plot indicating that cells of the smooth-swimming mutant occupied 76.5±13.9% of the channels visualized in the yellow fluorescence channel (n=596 channels). In separate experiments, non-motile cells occupied 0% of the channels visualized in the cyan fluorescence channel (n=537). The black solid circles and the white circles represent motile and non-motile cells in the plot, respectively. FIG. 6D is a cell density plot illustrating changes in the cell density per channel over one hour when the microfluidic device contained only motile cells. The means were calculated from data obtained from at least three different fields of view across the microfluidic device, over two biological replicates. The bars represent the standard deviation. FIG. 6E shows a composite image that displays an overlay of the phase and fluorescent (yellow) channels for a 1:1 mixture of non-motile (cyan) and motile (yellow) cells and its corresponding occupancy plot. Smooth-swimming cells expressing eYFP are visible mostly in the orthogonal channels, with a few cells remaining in the main trench. The non-motile cells expressing eCFP are exclusively visible in the main trench. In this mixture, 77.5±11.9% of the channels were occupied by the motile cells whereas 0.3±0.5% of the channels were occupied by non-motile cells (n=628 channels).

As recognized herein, orthogonal channels of a microfluidic device are selectively occupied by motile cells. In an example, wild-type E. coli cells were cultured and resuspended in motility buffer (MB) solutions at an optical density of 600 nanometers (nm) (OD₆₀₀) of 1.3-1.5. The cells were then introduced into the microfluidic chamber of a microfluidic device. The cells were observed on a phase microscope approximately 15 minutes after loading the cells. The microfluidic device of this example is a mother machine having a main trench flanked by several orthogonal channels measuring 25 micrometers (μm) in length. Each orthogonal channel is 1.4 μm in width and 1.5 μm in height.

Due to the substantial viscous resistance inside the channels, bacterial entry to the channels is generally hindered. Therefore, centrifugation has traditionally been utilized to force bacterial cells into the channels. However, it is recognized that that motile cells readily entered the orthogonal channels without any external forcing, as illustrated by FIG. 6A. Several distinct regions within the device were scanned and cells were observed as frequently entering and exiting the channels. As such, the present methods do not utilize a centrifugation step, instead utilizing the native motility of the cells for channel occupation.

Epifluorescence microscopy was employed to determine how effectively the motile cells occupied the orthogonal channels. A yellow fluorescent protein, eYFP, was expressed in the wild-type strain, which was then introduced into the microfluidic device. After 15 minutes, fluorescence images were taken by suitably illuminating the cells, as shown in FIG. 6B. The number of loaded and empty channels were counted in the images that were acquired from multiple regions within the device. It was observed that the motile cells frequently enter and exit the channels, such that approximately 68% of the channels were occupied by wild-type cells at any given instant, as shown in FIG. 6A.

The rotary flagella that propel a cell of E. coli generate a thrust force of about 0.5-1 piconewtons (pN). This force is adequate to penetrate the orthogonal channels in the microfluidic device. Nevertheless, the flagella can switch their direction of rotation from default counterclockwise (CCW) to clockwise (CW), which can reverse the direction of propulsive force inside constricted spaces. As a result, wild-type cells enter and escape constricted spaces readily, including the pores in soft-materials such as agar. Indeed, cells were observed exiting the orthogonal channels frequently, which increases challenges in monitoring the properties of single cells over longer durations.

Therefore, mutant cells lacking the ability to switch the direction of rotation of their flagella were examined to determine if the mutant cells were more likely to remain trapped in the channels. A variant of E. coli RP437 deleted for the cheY allele were examined as an option. CheY, in its phosphorylated form, is a response regulator that modulates flagellar switching. In its absence, the flagella rotate exclusively CCW and the cells swim smoothly without tumbling. As such, cYFP was expressed in this smooth-swimming strain and the cells were loaded into the microfluidic device. As before, the cells were illuminated and imaged with a sensitive scientific Complementary Metal-Oxide-Semiconductor (sCMOS) camera in the yellow channel. In a separate set of experiments, a AcheY strain was employed that expressed eCFP and was deleted for the fliC allele. FliC are flagellin proteins that assemble into the extracellular flagellar filaments, and in their absence, the cells are non-motile. These non-motile cells were loaded into the microfluidic device and imaged in the cyan channel. Both the motile and non-motile strains were adjusted OD₆₀₀ of 1.3 prior to loading into the microfluidic device.

Fluorescence imaging revealed that the smooth-swimming motile cells populated the orthogonal channels similar to the wild-type, with only a handful of cells remaining in the main trench, as shown in the left panel of FIG. 6B. In contrast, cells of the non-motile strain were exclusively observed in the main trench, as shown in the right panel of FIG. 6B. Remarkably, out of the several hundred channels that were scanned, almost no channels were occupied by the non-motile cells, as illustrated by the resulting occupancy plot of FIG. 6C. In comparison, about 77% of the channels were occupied by the smooth-swimming cells, which was slightly higher than the corresponding number for the wildtype. Thus, the smooth-swimming bacteria appear better at occupying the orthogonal channels of the microfluidic device than wild-type cells.

Considering that the smooth swimmers are better at occupying the orthogonal channels, it was determined whether the cells remain trapped in individual channels for long durations. For this evaluation, images were recorded 5, 10, 15, 30, and 60 minutes after loading the smooth-swimming cells in the microfluidic device. From the images, the time-varying changes in the cell density per channel were quantified by visually tracking the number of cells in each channel. As illustrated in the cell density plot of FIG. 6D, the cell density remained approximately constant at about 4 cells per channel. Additionally, several smooth-swimming cells that entered the channels remained trapped for the entire duration of observation.

Based on the orthogonal channels of FIG. 6B being occupied exclusively by the motile cells, it is recognized that the orthogonal channels can be used to separate a mixture of non-motile and smooth-swimming cells. For example, equal volumes of the two cultures were mixed and the final concentration was adjusted to an OD₆₀₀ of about 1.3-1.5. The mixture was loaded into the microfluidic device and imaged in the dual-fluorescence channels, with the resulting image shown in the left panel of FIG. 6E. The motile cells occupied about 77.5% of the total channels (n=628), whereas the non-motile cells occupied only 2 out of the total channels, as illustrated by the plot of the right panel of FIG. 6E. Accordingly, motility enables single cells to penetrate or traverse otherwise inaccessible interiors of the microfluidic device, with the depth of penetration only limited by the channel length, which is about 25 μm in the present example. In comparison, the entry of simply diffusing cells into orthogonal channels appears to be kinetically limited. As such, these experiments confirm that the presently disclosed microfluidic device has orthogonal channels that permit single-file movement of bacterial cells and therefore exclusively selects for motile cells while excluding non-motile cells. The device provides nearly 100% isolation of motile cells from a mixture of non-motile and motile cells. Such results are confirmed by single-cell fluorescence assays that reveal cells trapped in the micro-channels.

A variety of materials and methods were utilized in performing the above experiments and analyzing their results. Initial details are provided with respect to bacterial strains, cell culturing, and plasmids used herein. All experiments were performed with the E. coli RP437 strain or its derivatives, as shown in Table 1 below. Fresh colonies were streaked on Luria-Bertani (LB) agar plates from frozen glycerol stocks. Overnight cultures were grown in tryptone broth (TB) at 30° C. and 40 revolutions per minute (RPM in a rotating test-tube holder (Fischer Scientific) and diluted the cultures 1:100 times in 10 mL fresh TB the subsequent day to prepare day cultures. The day cultures were grown at 33° C. in a shaker incubator set at 170 RPM and the cells were harvested once they reached an optical density at 600 nm (OD₆₀₀) between 0.5-0.6, as measured with Thermo Spectronic 200 spectrophotometer. The media was supplemented with antibiotics (100 μg/mL ampicillin sodium salt, Sigma Aldrich) where appropriate and fluorescent proteins were expressed by adding 100 μM IPTG (isopropylthio-β-galactoside, Thermo Fischer Scientific). The fluorescent protein genes (cCFP and cYFP) were separately cloned in ptrc99A between EcoRI and XbaI cloning sites.

TABLE 1
Strain and plasmid information.

| No. | Strain or Plasmid | Function |
|---|---|---|
| 1 | ΔcheR-cheB, ΔfliM, ΔfliG | Motile strain |
| 2 | HCB 33 (RP437) + ΔCheY | Non-Motile strain |
| 3 | pTrc99A + eYFP (n-terminal fusion) | eYFP fluorescence |
| 4 | pTrc99A + eCFP | eCFP fluorescence |

To perform motility assays, the cell culture was washed twice in motility buffer (MB) at 1500 g for 5 minutes in a centrifugation unit (Scilogex SCT412). The supernatant was discarded, and the cell pellet was gently resuspended in an appropriate volume of MB to obtain the desired OD₆₀₀ value. Care was taken during washing and resuspension to prevent the breakage of flagellar filaments. MB was prepared with 0.01 M potassium phosphate, 0.067 M NaCl, 0.1 mM EDTA, 1 μM methionine, and 10 mM lactic acid, and adjusted to pH 7.0.

To perform fluorescence and phase imaging, a 60× oil immersion TIRF objective was used to obtain data associated with phase and fluorescence. The halogen light for phase imaging was filtered with a long-pass filter (720 LP, Chroma) to prevent blue wavelengths from adversely impacting cell physiology. Phase images were recorded with a CCD camera (UI-3240LE, IDS Imaging). Fluorescence images were collected in the epifluorescence mode on a Nikon TiE microscope. Cells expressing eCFP or eYFP were illuminated with a white-LED source (SOLA SE II 365 Light Engine, Lumencor), filtered appropriately: 435/20 ex and 480/40 em (Nikon Inc.) for CFP and ZT514/10, ET520 LP, and ET555/55m (Chroma Inc) for eYFP. Two-channel fluorescence detection was conducted in tandem on a sCMOS camera (Andor Zyla, Oxford Instruments) at 2 frames per second with an exposure time of 0.5 s. Phase images were analyzed with ImageJ to measure quantify cells and their movements. The empty or occupied channels were counted in fluorescence images to determine the number of occupied channels and cells per channel. The fluorescence images were analyzed with a custom-written MATLAB code that determined the fluorescence intensity (brightness) of a single cell. Contrast enhancement, background correction, image rotation, and false color were employed in ImageJ to generate representative images from the raw fluorescence data.

To collect video data of motility, cells grown as described above were diluted in TB and imaged in a culture dish (Delta T Culture Dish, Bioptechs) with a Nikon Ti-E microscope equipped with a 20× objective. Motility was recorded for a suitable time period with a charged-coupled device (CCD) camera (UI-3240LE, IDS Imaging) at 20 frames per second. Videos were analyzed using custom-written particle tracking MATLAB code to count the number of cells in the field of view. Moreover, all statistical analyses were performed with the Student's t test, with equal or unequal variances as appropriate using Prism 9.3.0 (GraphPad, USA).

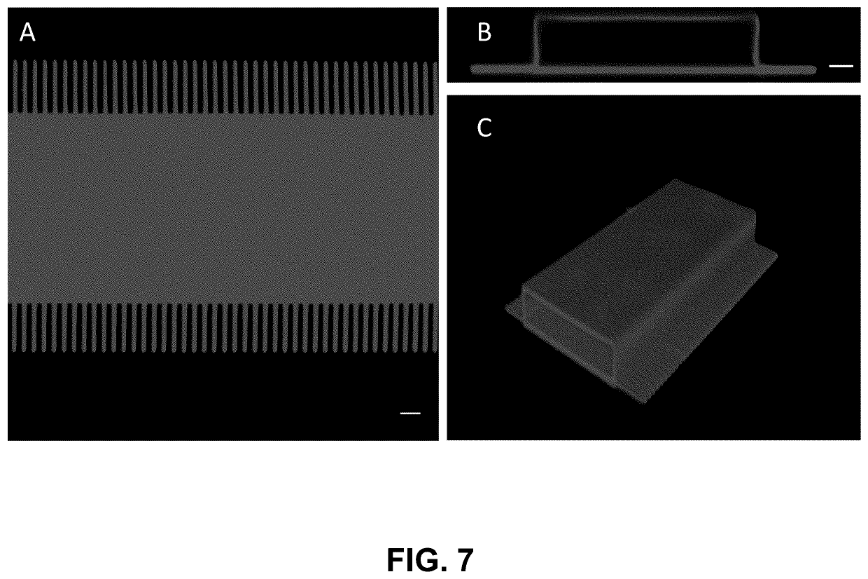

Certain experiments described herein utilized a microfluidic device having a central trench between two rows of orthogonal channels. FIGS. 7A-C show optical images of a microfluidic device or mother machine obtained via confocal microscopy. FIG. 7A is an x-y cross-section of the trench and the growth channels at the interface of PDMS and coverslip and FIG. 7B is a x-z reconstruction of 269 z-planes acquired along the height of the trench. The surface of the trench, growth channel and coverslip are stained using DiI, a lipophilic carbocyanine dye, and the provided scale is 10 μm. Additionally, FIG. 7C is an isometric three-dimensional (3D) rendition reconstructed from z-stacks and shows a section of the DiI-stained internal surfaces of mother machine. The microfluidic device can be efficiently used within the motility analysis system disclosed herein to isolate motile bacteria from a mixture of non-motile/motile bacteria. In some examples, the microfluidic device provides a separation efficiency of about 100%. In some examples, the microfluidic device provides a separation efficiency of at least about 95, 97.5, 99, or 99.5%.

To fabricate a microfluidic device as used herein, a CAD drawing of the main trench and growth channels was designed and generated in AutoCAD 2022 (AutoDesk, USA). The design was used to fabricate photomasks with a Heidelberg MLA-150 DWL 66 laser writer. The master silicon wafer was treated with O₂ plasma (Tepla M4L Asher) at 500 W and an O₂ flow rate of 600 standard cubic centimeters (cm) per minute. Next, 5 nm Titanium (Ti) and 100 nm Aluminum (Al) were sputter deposited for alignment marks with a Kurt L. Lesker CMS-18 Dual Chamber Sputterer. The marks were patterned with 2 μm thick layer of positive thin resist AZ 1512 (MicroChemicals, Germany), and etched using a JT Baker Aluminum etchant for Al and a 1% HF for Ti. A 1.5 μm thick layer of SU8 2002 was spin-coated at 4000 RPM, followed by soft baking of the wafer at 95° C. for 1 minute on a hotplate. The spin-coated channel layer was then exposed to 70 millijoules (mJ)/cm² of UV (Karl Suss MA6 mask aligner) light through a photomask generated for the growth channels. The wafer was soft baked for 1 minute at 95° C. After baking, the wafer was developed using a SU8 developer for 1 minute. Another 25 μm layer of SU-8 was spin-coated at 1600 RPM on the wafer using SU8 2015 and soft-baked at 95° C. for 5 minutes. The layer was exposed to 150 mJ/cm² UV light through the photomask generated for the main trench. Post-exposure, the wafer was baked at 95° C. for 5 minutes on the hotplate and developed using SU8 developer for 5 minutes. Finally, the master wafer was hard baked at 175° C. to anneal out the surface cracks.

A negative mold was prepared by casting polydimethylsiloxane (PDMS) onto the wafer. Briefly, PDMS (Sylgard 184, Dow Corning, Midland, MI, USA) was mixed at 10:1 (w/w) base-to-curing agent ratio, poured onto the patterned master wafer, and cured in an oven at 60° C. for 2 hours (h). The cured PDMS negative was peeled off the wafer and holes for the inlet and outlet were punched at the end of the channel using a 1 mm biopsy punch (P125, Acuderm Inc., Ft. Lauderdale, FL, USA). The negative was then plasma treated for 2 minutes in a 400W plasma cleaner (PE-25; Plasma Etch), followed by bonding it to a 35 mm No. 1.5 glass bottom dish (FD35, WPI, Sarasota, FL, USA) or thermal dish (Delta T Heated Culture Dish, Bioptechs). The dish was then baked at 90° C. for 15 minutes to permanently attach PDMS to the glass. The dimensions of the main trench were validated to be 15 mm (L)×100 μm (W)×25 μm (H) and the growth channels were 25 μm (L)×1.4 μm (W)×1.5 μm (H) using SEM and confocal.

To operate the microfluidic device, the device was passivated with 20 mg/mL bovine serum albumin (VWR Life sciences) for 1 h at room temperature prior to use. Before each experiment, the air was removed from the device by filling the interiors with MB using a syringe (BD 3 mL) attached to a metal tube (New England small tube corporation 0.28″ OD×0.016″ ID). Approximately 200-300 microliters (μL) of the washed bacteria were then aspirated into a syringe and connected to the inlet port of the device with a 7-10 cm long polyethylene tubing (0.58 mm inner diameter). Cells were loaded into the main channel from the syringe with a syringe pump (Fusion 200, Chemyx) operated at a flow rate of 200 μL/h. The main trench was visually scanned on a Nikon Ti-E phase microscope with a 20× phase objective. The flow was stopped once the cells were detected in the main trench. To study regrowth, the tolerant cells were loaded using same method as above. Once the appropriate number of cells entered the channels, introduction of cell suspension was stopped and switched to liquid LB at a flow rate of 250 μL/h. The liquid LB was continuously perfused for several hours, and the microfluidic device was maintained at 37° C. during the experiment. An automated stage (MS-2000, Applied Scientific Instrumentation) was utilized to repeatedly cycle between channels in different regions and videos were recorded in phase.

As discussed herein, a variety of industries including medical, food and crop, and water management industries can benefit from use of the presently disclosed motility analysis system for identifying and monitoring pathogenic and non-pathogenic bacteria in human, plant, or animal hosts. Indeed, the motility analysis system can be used to identify any disease-causing bacteria in plants, animals, and humans that uses any form of motility. For example, bacteria that cause gastrointestinal infections (such as E. coli, Salmonella, Clostridium difficile, and Vibrio cholerae), urinary tract infections (such as Pseudomonas aeruginosa, E. coli, and Proteus mirabilis), stomach infections (such as Helicobacter pylori), meningitis (such as Neisseria meningitidis), skin infections (such as Vibrio vulnificus), sexually transmitted diseases caused by bacteria (such as Neisseria gonorrhoeae), as well as eye and lung infections (such as Bacillus cereus and Pseudomonas aeruginosa). These methods can be utilized to detect bacterial pathogens in soil and plants, such as a motile bacterial species that infects fruits and plants is Erwinia amylovora. This species causes Fire blight, a deadly infection in apples, pears, and other plant products, leading to millions of dollars of losses each year. These methods also enable detection of Burkholderia glumae, a motile bacterial pathogen that infects rice, which causes bacterial panicle blight. The motility analysis system can also detect other bacterial pathogens, such as Candidatus liberibacter asiaticus and Candidatus liberibacter spp, which cause the devastating infections that have significantly harmed Florida's citrus industry.

The motility analysis system provides pathogenic bacteria phenotyping and antibiotic susceptibility testing. The motility analysis system implements a label-free technique based on bacterial morphology that can be obtained with low-cost objective lenses. As such, the motility analysis system is capable of processing any fluid sample to identify one or more pathogens therein in seconds to a few minutes using machine learning/artificial intelligence. The motility analysis system does not require laboratories or dedicated personnel, and does not involve the substantial capital outlays typical of contemporary bacterial genotyping or phenotyping techniques. The motility analysis system can therefore be deployed at minimal costs throughout various industries, including healthcare, agriculture, livestock, travel, water management, and oil and gas

The motility analysis system can accurately identify bacterial pathogens in any sample over a short testing period via unlimited testing, where detection is inexpensive and limited only by the sample availability. This accessibility therefore enhances monitoring and critical epidemiological insights during epidemics and enables evaluation of any antibiotic resistance in pathogens to help detect superbugs rapidly. Moreover, certain examples include a relatively straightforward portable imaging device, which can be rapidly deployed in any sector for which detecting pathogens is desirable.

Other objects, features, and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from the detailed description. In further embodiments, features from specific embodiments may be combined with features from other embodiments. For example, features from one embodiment may be combined with features from any of the other embodiments. In further embodiments, additional features may be added to the specific embodiments described herein.

What is claimed is:

1. A motility analysis system, comprising:
a memory storing processor-executable instructions; and
one or more processors communicatively coupled to the memory and configured to execute the processor-executable instructions from the memory, the one or more processors configured, when executing the instructions, to:
receive video data of a fluid sample containing bacteria captured through a microscope;
generate a two-dimensional (2D) time-projection of motile tracks of the bacteria based on a pixel-by-pixel analysis of the video data;
process the 2D time-projection of the motile tracks with a quantitative analysis model to identify a match between the 2D time-projection of the motile tracks and a plurality of predetermined 2D time-projections; and
output data indicative of an identity of the bacteria in response to identifying the match.

2. The motility analysis system of claim 1, wherein the one or more processors are configured, when executing the instructions, to generate the 2D time-projection by:
determining a pixel characteristic based on a time-varying intensity of each pixel in the video data over a duration of the video data; and
reshaping the pixel characteristic of each pixel to an original image size of the video data to generate the 2D time-projection.

3. The motility analysis system of claim 1, wherein the plurality of predetermined 2D time-projections are labeled with a corresponding plurality of bacteria samples, and wherein the quantitative analysis model is trained on the plurality of predetermined 2D time-projections to distinguish and identify motile bacteria via machine learning.

4. The motility analysis system of claim 1, wherein the one or more processors are further configured, when executing the instructions, to:
receive additional video data of the fluid sample captured after application of an antibacterial compound to the fluid sample;
generate an additional 2D time-projection of motile tracks of the bacteria; and
output data indicative of a resistance of the bacteria to the antibacterial compound based on a comparison between the 2D time-projection and the additional 2D time-projection.

5. The motility analysis system of claim 1, wherein the one or more processors are further configured, when executing the instructions, to:
query a database of a plurality of bacterial treatments based on the identity of the bacteria; and
output data indicative of a selected bacterial treatment based on the query.

6. The motility analysis system of claim 1, wherein the one or more processors are further configured, when executing the instructions, to:
receive user input indicative of an accuracy of the identity of the bacteria; and
train the quantitative analysis model based on the 2D time-projection and the accuracy of the identity of the bacteria.

7. The motility analysis system of claim 1, comprising:
a microscope attachment configured to couple to a computing device having a camera to capture the video data, wherein the microscope attachment comprises:
a microscope lens to magnify images captured through the camera;
a light source to illuminate the fluid sample; and
a sample retaining device configured to receive an observation retainer in view of the camera and the microscope lens.

8. The motility analysis system of claim 7, wherein the observation retainer comprises a microfluidic device having a first trench, a second trench, and a plurality of orthogonal channels fluidly coupling the first trench and the second trench, and wherein the plurality of orthogonal channels is sized to isolate a motile fraction of the bacteria in the first trench or the second trench for generation of the 2D time-projection.

9. The motility analysis system of claim 7, wherein the observation retainer comprises a microscope glass slide.

10. The motility analysis system of claim 1, wherein the memory, the one or more processors, and a camera to capture the video are operatively coupled together within a smartphone or a tablet.

11. A method to identify a bacteria based on motility analysis, the method comprising:
receiving video data of a fluid sample containing bacteria captured through a microscope;
generating a two-dimensional (2D) time-projection of motile tracks of the bacteria based on a pixel-by-pixel analysis of the video data;
processing the 2D time-projection of the motile tracks with a quantitative analysis model to identify a match between the 2D time-projection of the motile tracks and a plurality of predetermined 2D time-projections; and
transmitting data indicative of an identity of the bacteria in response to identifying the match.

12. The method of claim 11, wherein the step of generating the 2D time-projection further comprises:
determining a pixel characteristic based on a time-varying intensity of each pixel in the video data over a duration of the video data; and
reshaping the pixel characteristic of each pixel to an original image size of the video data to generate the 2D time-projection.

13. The method of claim 11, wherein the plurality of predetermined 2D time-projections are labeled with a corresponding plurality of bacteria samples, and wherein the quantitative analysis model is trained on the plurality of predetermined 2D time-projections to distinguish and identify motile bacteria via machine learning.

14. The method of claim 11, further comprising the steps of:
receiving additional video data of the fluid sample captured after application of an antibacterial compound to the fluid sample;
generating an additional 2D time-projection of motile tracks of the bacteria; and
transmitting data indicative of a resistance of the bacteria to the antibacterial compound based on a comparison between the 2D time-projection and the additional 2D time-projection.

15. The method of claim 11, further comprising the steps of:
querying a database of a plurality of bacterial treatments based on the identity of the bacteria; and
transmitting data indicative of a selected bacterial treatment based on the query.

16. The method of claim 11, further comprising the steps of:
receiving a user input indicative of an accuracy of the identity of the bacteria; and
training the quantitative analysis model based on the 2D time-projection and the accuracy of the identity of the bacteria.

17. The method of claim 11, further comprising:
coupling a microscope attachment to a computing device having a camera to capture the video data, wherein the microscope attachment contains a sample retaining device configured to receive an observation retainer.

18. The method of claim 17, wherein the observation retainer comprises a microfluidic device having a first trench, a second trench, and a plurality of orthogonal channels fluidly coupling the first trench and the second trench.

19. The method of claim 18, wherein the plurality of orthogonal channels is sized to isolate a motile fraction of the bacteria in the first trench or the second trench for generation of the 2D time-projection.

20. The method of claim 17, wherein the observation retainer comprises a microscope glass slide.
